(19) United States
(12) Patent Application Publication
(10) Pub. No.: US 2025/0255941 A1
(43) Pub. Date: Aug. 14, 2025

(54) COMPOSITION FOR AMELIORATING HAIR LOSS COMPRISING BOTULINUM-DERIVED PEPTIDE

(71) Applicant: BPMED CO., LTD., Seoul (KR)

(72) Inventor: Ki Young YOON, Seoul (KR)

(21) Appl. No.: 18/703,591

(22) PCT Filed: Jul. 6, 2022
(86) PCT No.: PCT/KR2022/009761

§ 371 Date: Apr. 22, 2024

(30) Foreign Application Priority Data
Oct. 22, 2021 (KR) 10-2021-0141684
Jun. 28, 2022 (KR) 10-2022-0079197

(51) Int. Cl. A61K 38/48; A61K 8/66; A61K 47/64; A61K 47/66; A61P 17/14; A61Q 7/00

(52) U.S. Cl. CPC A61K 38/4893; A61K 8/66; A61K 47/64; A61K 47/66; A61P 17/14; A61Q 7/00; C12Y 304/24069

(57) ABSTRACT

The present invention relates to a botulinum-derived peptide (botulinum toxin recombinant protein). Since the botulinum toxin recombinant protein according to the present invention can be easily delivered transdermally through fusion with a cell-penetrating peptide and promote the proliferation of dermal papilla cells and enhance prostaglandin F₂ₐ expression to promote hair growth in hair loss areas, thereby ameliorating alopecia, it can be effectively used for preventing, ameliorating or treating alopecia in fields such as cosmetics and pharmaceuticals.

### TECHNICAL FIELD

The present invention relates to a composition for ameliorating alopecia comprising a botulinum-derived peptide (botulinum toxin recombinant protein).

The present invention claims priority based on Korea Patent Application No. 10-2021-0141684 filed on Oct. 22, 2021 and Korea Patent Application No. 10-2022-0079197 filed on Jun. 28, 2022, and all contents disclosed in the specifications and drawings of the applications are incorporated into the present application.

### BACKGROUND ART

Human hair is very important in our lives not only because it protects our skin and scalp, but also for social and sexual communication. Hair is composed of keratin protein and grows from follicles in the dermis. Hair growth exhibits a certain cycle, which is divided into anagen (growth phase), catagen (regression or transition phase), and telogen (resting phase). Scalp follicles are composed of dermal papilla cells, keratinocytes, inner and outer root sheath cells, and melanocytes. Human hair follicle dermal papilla cells (HFDPCs) are one of special fibroblast cells and participate in the morphogenesis of follicles. Since HFDPCs are related to hair growth, changes in HFDPC transcription factors and cytokines are important. Keratin protein is composed from human keratinocytes and is the main tissue of hair. Recently, factors involved in cell growth (proliferation), degradation, and apoptosis in scalp follicles during the anagen phase have been revealed by various molecular analysis systems. It is known that extracellular signal-regulated kinase (ERK) and serine/threonine protein kinase (AKT) pathways are involved in cell proliferation of scalp dermal papilla. It is well known that the ERK signaling pathway plays a certain role in mitogenesis or cell growth, and it has been reported that AKT plays an important role in mediating survival signals.

Normal people have a lot of hair in the growth phase, whereas people with alopecia have a lot of hair in the resting phase, resulting in visible hair loss. As alopecia progresses, the growth phase becomes shorter, and as a result, hair becomes increasingly smaller. Therefore, to treat alopecia, it is important to allow follicles in the resting phase to quickly move to the growth phase and to lengthen the shortened growth phase.

Currently, there are two types of hair growth promoters approved by the FDA: minoxidil (transdermally applied drug) and finasteride (orally administered drug). Minoxidil was used as an oral antihypertension medication, but it was observed that hirsutism appeared in patients taking this medication, so it is currently being used for scalp application to treat androgenetic alopecia. Minoxidil is a pyrimidine derivative that dilates blood vessels in the scalp to locally increase blood flow and activates hair matrix cells to slow down alopecia and promote the growth of downy hair. Therefore, it is currently widely used as an alopecia therapeutic agent. Finasteride, which is the first orally administered therapeutic agent for treating androgenetic alopecia, prevents alopecia and promotes hair growth by inhibiting type IIα-reductase. Since it was approved by the FDA in 1997, finasteride is currently used by about 2.6 million people worldwide.

However, minoxidil has been reported to have side effects such as weight gain, edema, increased heart rate, angina, dermatitis, and itching, and in the case of finasteride, side effects such as male sexual dysfunction have been reported in clinical cases. Therefore, the use of these drugs is limited or patients themselves show aversion to these drugs. Accordingly, there is increasing consumer interest in safe alopecia prevention and hair growth-promoting substances, and research on this is also being actively conducted.

Meanwhile, botulinum toxin is a neurotoxin protein produced by Clostridium botulinum and is reported to inhibit the secretion of acetylcholine and catecholamine, which are neurotransmitters in neurons. There are eight types of botulinum toxin: types A, B, C, D, E, F, G, and H, and types A and B are commercially used.

Botulinum toxin is a neurotoxin protein, and the median lethal dose for humans is 1.3 to 2.1 ng/kg when injected intravenously or intramuscularly and 10 to 13 ng/kg when inhaled, indicating that it is a highly toxic substance. However, when adjusted to an appropriate amount, it can be used as a drug for treating neurological disorders, muscle diseases, hyperhidrosis, square jaw or the like, and it is most often used for cosmetic purposes such as reducing wrinkles and calf muscles.

Since botulinum toxin is a very large molecule having a molecular weight of 150 kDa with combined light and heavy chains, it is difficult to penetrate the skin and is therefore only used through injections. The toxin lasts for 3 to 6 months, and thus the toxin requires regular treatments. Therefore, many studies are being conducted to find another effective delivery means capable of providing user convenience, but the results are still insufficient.

### DISCLOSURE

### Technical Problem

The present inventors confirmed that a botulinum toxin recombinant protein (skin-penetrating botulinum-derived ingredient peptide) according to the present invention promotes the proliferation of dermal papilla cells and expression of prostaglandin F₂ₓ and that alopecia was ameliorated when it was applied to hair loss areas, and thereby completed the present invention.

Therefore, an object of the present invention is to provide a pharmaceutical composition for ameliorating alopecia, comprising a botulinum toxin recombinant protein as an active ingredient, wherein in the botulinum toxin recombinant protein, a cell-penetrating peptide consisting of an amino acid sequence of SEQ ID NO: 1 is fused to one end or both ends of a botulinum toxin light chain.

Another object of the present invention is to provide a quasi-drug composition for ameliorating alopecia, comprising the botulinum toxin recombinant protein as an active ingredient.

Still another object of the present invention is to provide a composition for external skin application for ameliorating alopecia, comprising the botulinum toxin recombinant protein as an active ingredient.

Yet another object of the present invention is to provide a cosmetic composition for ameliorating alopecia, comprising the botulinum toxin recombinant protein as an active ingredient.

However, the technical problems to be solved by the present invention are not limited to the problems mentioned above, and other problems not mentioned may be clearly understood from the description below by those skilled in the art to which the present invention pertains.

### Technical Solution

One aspect of the present invention provides a pharmaceutical composition for ameliorating alopecia, comprising a botulinum toxin recombinant protein as an active ingredient, wherein in the botulinum toxin recombinant protein, a cell-penetrating peptide consisting of an amino acid sequence of SEQ ID NO: 1 is fused to one end or both ends of a botulinum toxin light chain.

Another aspect of the present invention provides a quasi-drug composition for ameliorating alopecia, comprising the botulinum toxin recombinant protein as an active ingredient.

Still another aspect of the present invention provides a composition for external skin application for ameliorating alopecia, comprising the botulinum toxin recombinant protein as an active ingredient.

Yet another aspect of the present invention provides a cosmetic composition for ameliorating alopecia, comprising the botulinum toxin recombinant protein as an active ingredient.

In one embodiment of the present invention, the botulinum toxin recombinant protein may consist of one or more amino acid sequences selected from the group consisting of SEQ ID NO: 31 to SEQ ID NO: 58, but is not limited thereto.

In another embodiment of the present invention, the botulinum toxin light chain may consist of one or more amino acid sequences selected from the group consisting of SEQ ID NO: 3 to SEQ ID NO: 9, but is not limited thereto.

In still another embodiment of the present invention, the botulinum toxin light chain may further include a hexahistidine tag at one end, but is not limited thereto.

In yet another embodiment of the present invention, the botulinum toxin light chain may be selected from the group consisting of botulinum toxin serotypes A, B, C, D, E, F, and G, but is not limited thereto.

In yet another embodiment of the present invention, the cell-penetrating peptide may be fused to a carboxyl terminus, an amino terminus, or both of the botulinum toxin light chain, but is not limited thereto.

In yet another embodiment of the present invention, the fusion may be achieved by a peptide bond or a covalent bond.

In yet another embodiment of the present invention, the composition may promote the proliferation of dermal papilla cells, but is not limited thereto.

In yet another embodiment of the present invention, the composition may promote prostaglandin F₂ₐ expression, but is not limited thereto.

In yet another embodiment of the present invention, the composition may promote hair growth and reduce alopecia, but is not limited thereto.

In yet another embodiment of the present invention, the composition may satisfy one or more of the following characteristics, but is not limited thereto:
(a) increased hair thickness; (b) increased hair density (reduction in number of lost hairs); (c) inhibition of sebum secretion in the scalp; (d) improved scalp hygiene; and (e) reduced hair sinking.

In yet another embodiment of the present invention, the composition for ameliorating alopecia may be for transdermal administration, but is not limited thereto.

In addition, the present invention provides an alopecia prevention or treatment method, comprising administering a composition comprising the botulinum toxin recombinant protein as an active ingredient to a subject in need thereof.

In addition, the present invention provides a use of a composition comprising the botulinum toxin recombinant protein as an active ingredient for preventing or treating alopecia.

In addition, the present invention provides a use of the botulinum toxin recombinant protein for preparing a drug for treating alopecia.

In addition, the present invention provides a method of ameliorating alopecia, comprising administering a composition comprising the botulinum toxin recombinant protein as an active ingredient to a subject in need thereof.

In addition, the present invention provides a use of a composition comprising the botulinum toxin recombinant protein as an active ingredient for ameliorating alopecia

In addition, the present invention provides a use of the botulinum toxin recombinant protein for preparing a drug for ameliorating alopecia.

In addition, the present invention provides a pharmaceutical composition for promoting hair growth comprising the botulinum toxin recombinant protein as an active ingredient.

In addition, the present invention provides a method for promoting hair growth comprising administering a composition comprising the botulinum toxin recombinant protein as an active ingredient to a subject in need thereof.

In addition, the present invention provides a use of a composition comprising the botulinum toxin recombinant protein as an active ingredient for promoting hair growth.

In addition, the present invention provides a use of the botulinum toxin recombinant protein for preparing a drug for promoting hair growth.

### Advantageous Effects

Since a botulinum toxin recombinant protein according to the present invention can be easily delivered transdermally through fusion with a cell-penetrating peptide and promote the proliferation of dermal papilla cells and enhance prostaglandin F₂ₐ expression to reduce hair loss and promote hair growth, thereby ameliorating alopecia, it can be effectively used for preventing, ameliorating or treating alopecia in fields such as cosmetics and pharmaceuticals.

### DESCRIPTION OF DRAWINGS

FIG. 1 is a diagram showing the results of measuring absorbance after treating human papilla cells with a botulinum toxin recombinant protein.

FIG. 2 is a diagram showing the results of measuring absorbance after treating human papilla cells with fetal bovine serum (FBS) as a positive control for a botulinum toxin recombinant protein.

FIG. 3 is a diagram showing the results of measuring cell viability after treating human papilla cells with a botulinum toxin recombinant protein.

FIG. 4 is a diagram showing the results of measuring cell viability after treating human papilla cells with FBS as a positive control for a botulinum toxin recombinant protein.

FIG. 5 is a diagram showing a standard curve obtained by applying standard prostaglandin F₂ₐ according to the amount to analyze the expression level of prostaglandin F₂ₓ in keratinocytes.

FIG. 6 is a diagram showing the results of analyzing the absorbance value of prostaglandin F₂ₐ expression measured in keratinocytes.

FIG. 7 is a diagram showing the results of evaluation of an alopecia ameliorating effect evaluated by subjects and evaluators over time after administering a composition according to the present invention to various alopecia patients.

FIGS. 8 to 11 are diagrams showing the results of confirming an alopecia ameliorating effect over time after administering a composition according to the present invention to various alopecia patients.

### BEST MODE

The present inventors confirmed that a botulinum toxin recombinant protein (skin-penetrating botulinum-derived ingredient peptide) obtained by fusing a botulinum toxin light chain to a cell-penetrating peptide promotes the proliferation of dermal papilla cells and enhances the expression of prostaglandin F₂ₐ to ameliorate alopecia, resulting in reduced hair loss and promotion of hair growth, and thereby completed the present invention.

Since a botulinum toxin recombinant protein according to the present invention can be easily delivered transdermally through fusion with a cell-penetrating peptide and promote the proliferation of dermal papilla cells and enhance prostaglandin F₂ₐ expression to reduce hair loss and promote hair growth, thereby ameliorating alopecia, it can be effectively used for preventing, ameliorating or treating alopecia in fields such as cosmetics and pharmaceuticals.

Hereinafter, the present invention will be described in detail.

Botulinum toxin is expressed as a single polypeptide, but after expression, through a reconstitution process, it is divided into a heavy chain (H chain) of about 100 kDa and a light chain (L chain) of about 50 kDa, and the H chain and the L chain are connected by a disulfide bond. The H chain binds to a receptor on a neuron and allows botulinum toxin to enter the inside through endocytosis. After the L chain of botulinum toxin enters a cell, it exits the endosome, enters the cytoplasm, and cleaves a soluble N-ethylmaleimide-sensitive factor attachment protein receptor (SNARE) protein in the cytoplasm, inhibiting acetylcholine secretion and thereby exhibiting a muscle-paralyzing effect. Therefore, inhibition of acetylcholine secretion from neurons is possible with the L chain alone, and the H chain and L chain may function independently.

However, a separated botulinum toxin light chain with a molecular weight of 50 kDa is unable to penetrate the cell membrane, and so it cannot function on its own. In general, in order for a botulinum toxin light chain to be delivered to the cytoplasm of a neuron to exhibit botulinum toxin-specific activity, the help of a botulinum toxin heavy chain of about 100 kDa is essential.

Therefore, the present invention provides a pharmaceutical composition for ameliorating alopecia, including a botulinum toxin recombinant protein as an active ingredient that allows a botulinum toxin light chain, which is not easily introduced into cells, to be delivered into cells with high efficiency by imparting cell-penetrating properties by fusing a cell-penetrating peptide to the botulinum toxin light chain, and in the botulinum toxin recombinant protein, a cell-penetrating peptide consisting of an amino acid sequence of SEQ ID NO: 1 is fused to one end or both ends of a botulinum toxin light chain.

In the present specification, “alopecia” refers to a phenomenon in which hair falls out from the scalp or a state in which hair becomes sparse or thin and may include all types of alopecia classified as alopecia in the art, except for cicatricial (scarring) alopecia. For example, the alopecia may be one or more selected from the group consisting of alopecia areata, androgenetic alopecia, tinea capitis, hypotrichosis, hereditary hypotrichosis simplex, circumscribed alopecia, congenital alopecia, alopecia pubis, alopecia seborrheica, alopecia senilis, alopecia totalis, alopecia universalis, and telogen effluvium, but is not limited thereto.

The term “hair growth” refers to growing hair. An effect of a composition according to the present invention may include an effect of promoting hair growth.

According to one embodiment of the present invention, it was confirmed that a botulinum toxin recombinant protein according to the present invention may promote the proliferation of dermal papilla cells, promote prostaglandin F₂ₓ expression in keratinocytes, promote hair growth, and reduce alopecia (Examples 1 to 3).

In addition, according to one embodiment of the present invention, it was confirmed that a composition including a botulinum toxin recombinant protein according to the present invention satisfies one or more of the following characteristics, thereby ameliorating pre-symptoms of alopecia and preventing, ameliorating, or treating alopecia (see Example 3):
(a) increased hair thickness;
(b) increased hair density (reduction in number of lost hairs);
(c) inhibition of sebum secretion in the scalp;
(d) improved scalp hygiene; and
(e) reduced hair sinking.

The composition of the botulinum toxin recombinant protein (skin-penetrating botulinum-derived ingredient peptide) in the present invention are the same as Korea Patent No. 10-1882461, and all contents disclosed in the above document are incorporated by reference in the present application.

In the present invention, “botulinum toxin recombinant protein” includes a cell-penetrating peptide and a botulinum toxin light chain and refers to a complex formed by a chemical bond such as peptide bond or covalent bond. Specifically, the botulinum toxin recombinant protein according to the present invention is capable of delivering the botulinum toxin light chain into cells with high efficiency by imparting cell-penetrating properties by fusing a cell-penetrating peptide to the botulinum toxin light chain, which is a macromolecule that is not easily introduced into cells, and at this time, the cell-penetrating peptide may be fused to a carboxyl terminus, an amino terminus, or both of the botulinum toxin light chain.

The botulinum toxin recombinant protein according to the present invention may be delivered into cells with high efficiency through the fusion of the botulinum toxin light chain and a cell-penetrating peptide, and the activity and stability of the botulinum toxin light chain are improved to maximize the inherent efficacy of the botulinum toxin in vivo.

In the present invention, “botulinum toxin” refers to any known type of botulinum toxin, whether subsequently discovered or not, including a variant or a fusion protein produced by bacteria or engineered by a recombinant technique.

In the present invention, the botulinum toxin light chain may be selected from the group consisting of botulinum toxin serotypes A, B, C, D, E, F, and G, and at this time, the botulinum toxin light chain may consist of one or more amino acid sequences selected from the group consisting of SEQ ID NO: 3 to SEQ ID NO: 9. In addition, the botulinum toxin light chain may consist of one amino acid sequence selected from the group consisting of SEQ ID NO: 3 to SEQ ID NO: 9. At this time, the botulinum toxin light chain may be encoded by a polynucleotide consisting of a base sequence selected from the group consisting of SEQ ID NOS: 10 to 16, but is not limited thereto.

In addition, the botulinum toxin light chain may further include a hexahistidine tag at one end. In the present invention, the form further including a hexahistidine tag at one end of the botulinum toxin light chain may consist of an amino acid sequence selected from the group consisting of SEQ ID NO: 17 to SEQ ID NO: 23, and it may be encoded by a base sequence selected from the group consisting of SEQ ID NO: 24 to SEQ ID NO: 30, but is not limited thereto.

In the present invention, the botulinum toxin light chain may alternatively be a botulinum toxin derivative, that is, a compound having botulinum toxin activity but optionally having one or more modifications in a part or sequence. For example, compared to the seven serotypes of the botulinum toxin light chain protein, it may be a form modified in a way that maintains the endopeptidase activity of the light chain while simultaneously enhancing properties or reducing side effects thereof by performing methods such as deletion, modification, replacement, and chimeric fusion on an amino acid sequence. Alternatively, a botulinum toxin light chain or a part of a botulinum toxin light chain produced by recombinant or chemical synthesis may be used.

In the present invention, the botulinum toxin recombinant protein may consist of one or more amino acid sequences selected from the group consisting of SEQ ID NO: 31 to SEQ ID NO: 58, and a polynucleotide encoding the amino acid sequences may be selected from the group consisting of SEQ ID NO: 59 to SEQ ID NO: 86, but is not limited thereto.

In addition, in the present invention, the botulinum toxin recombinant protein may consist of one amino acid sequence selected from the group consisting of SEQ ID NO: 31 to SEQ ID NO: 58.

According to one embodiment of the present invention, the botulinum toxin recombinant protein may preferably consist of an amino acid sequence represented by SEQ ID NO: 45, and a polynucleotide encoding the amino acid sequence may be a nucleotide sequence represented by SEQ ID NO: 73, but is not limited thereto.

SEQ ID NO: 45:
MKAMININKFLNQCPFVNKQFNYKDPVNGVDIAYIKIPNAGQMQPVKAF
KIHNKIWVIPERDTFTNPEEGDLNPPPEAKQVPVSYYDSTYLSTDNEKD
NYLKGVTKLFERIYSTDLGRMLLTSIVRGIPFWGGSTIDTELKVIDTNC
INVIQPDGSYRSEELNLVIIGPSADIIQFECKSFGHEVLNLTRNGYGST
QYIRFSPDFTFGFEESLEVDTNPLLGAGKFATDPAVTLAHELIHAGHRL
YGIAINPNRVFKVNTNAYYEMSGLEVSFEELRTFGGHDAKFIDSLQENE
FRLYYYNKFKDIASTLNKAKSIVGTTASLQYMKNVFKEKYLLSEDTSGK
FSVDKLKFDKLYKMLTEIYTEDNFVKFFKVLNRKTYLNFDKAVFKINIV
PKVNYTIYDGFNLRNTNLAANFNGQNTEINNMNFTKLKNFTGLFEFYKL
LCVRGIITSKTKSLDKGYNKLEHHHHHH
SEQ ID NO: 73:
atgaaggccatgatcaatattaacaagttcttaaatcaatgtccctttg
tcaacaaacagttcaactacaaggacccagttaatggagtagacatcgc
atatatcaagattcccaacgctggccagatgcaacccgttaaggcattt
aaaatccataacaaaatctgggttatcccagagcgggataccttcacca
accccgaggagggcgatctgaaccccccgccggaggcgaagcaggtccc
agtgagctactacgatagcacctacctcagcaccgacaacgagaaggac
aactacctcaaaggagtcacgaagttgttcgagagaatctactccacag
acctcggccgcatgcttctaaccagcattgtgcgtggcattcccttttg
gggcggctctaccatcgacacagagctgaaggtgatagacaccaactgc
atcaacgtaatccagcctgacggcagctaccgaagcgaggagcttaacc
tggtgatcatcggcccttccgccgatatcatccaattcgagtgcaagag
cttcggccacgaggtcctgaacctcacccggaacggctatggaagcacc
cagtacataagattcagccctgacttcaccttcgggtttgaggagagct
tggaggtcgacacaaaccccctgctgggagccgggaagttcgccactga
cccagccgtgactctggcacacgagctgatccacgccggtcaccgcctg
tacggcatagctataaacccaaacagggtgttcaaagtgaacaccaacg
cttactatgaaatgagcggcctggaggtgagcttcgaggagctgagaac
gttcgggggacatgatgctaaatttatcgacagcctgcaggagaacgag
ttcaggctgtactactacaataagttcaaggatatagcgagcactctga
acaaggccaagtccatcgtaggcactactgcatccctccagtatatgaa
gaatgtgttcaaagagaaatacctgctgagcgaggataccagcggtaag
ttcagcgtggataagcttaagttcgacaagctgtataagatgctcaccg
aaatctacaccgaggataatttcgttaagttcttcaaggtcctgaaccg
gaagacctacctgaacttcgacaaggccgtgttcaagatcaacatcgtg
cctaaagtgaactacaccatctacgacgggtttaacctgaggaacacca
acctggccgctaacttcaacgggcagaacacagagatcaacaacatgaa
tttcacgaagttgaagaacttcaccggactgtttgagttctacaaattg
ctgtgtgtgcgcgggatcatcactagcaagaccaagagccttgacaaag
gctacaacaagtgactcgagcaccaccaccaccaccactga

In the present invention, the cell-penetrating peptide (Macromolecule Transduction Domain; MTD) consisting of an amino acid sequence of SEQ ID NO: 1 may be a peptide that may mediate intracellular transport of a biologically active molecule and may have permeability with respect to both human skin keratinocytes and neurons, but is not limited thereto.

The cell-penetrating peptide preferably has no defined enzymatic or therapeutic biological activity, but serves as a carrier allowing intracellular transport across the cell membrane. It may be attached to an N-terminus or C-terminus and both termini of the cargo to be transferred into the cell, and it may be attached in a forward direction or a reverse direction at each terminus. In addition, the peptide according to the present invention is preferably applied as a monomer, but is not limited thereto, and it may also be used in the form of a dimer or polymer. Furthermore, the peptide according to the present invention may be a peptide including an amino acid sequence of SEQ ID NO: 1 as a minimum unit.

In the present invention, the cell-penetrating peptide may be encoded by a polynucleotide consisting of a base sequence of SEQ ID NO: 2, but is not limited thereto.

SEQ ID NO: 1:
KAMININKFLNQC
SEQ ID NO: 2:
aaggcgatga taaacataaa caagttcctg aaccagtgc

In the present specification, “active ingredient” refers to an ingredient that may exhibit desired activity alone or in combination with a carrier that is inactive in itself.

According to one embodiment of the present invention, the most appropriate administration route for the composition according to the present invention to exhibit an alopecia-ameliorating effect is absorption through the skin, and therefore, the composition according to the present invention may preferably be for transdermal administration and may be administered by methods such as direct application or dispersion to hair or the scalp.

“Hair” to which the composition of the present invention is applied includes hair roots and follicles of the head, hair on the head, eyelashes, eyebrows, beards, armpit hair, pubic hair, and all parts of the body with hair roots and follicles.

In the present invention, the composition according to the present invention may further include a transdermal absorption enhancer, but is not limited thereto.

The “transdermal absorption enhancer” is an ingredient among emulsifiers that affects skin penetration and is commonly used in transdermal patches. In the present invention, it enhances skin penetration and cellular penetration of the botulinum toxin recombinant protein and may be lecithin, lauryl pyrrolidone, glycerol monooleate, glycerol monolaurate, propylene glycol monolaurate, polyoxyethylene sorbitan monooleate, polyoxyethylene sorbitan monostearate, polyoxyethylene sorbitan monolaurate, sorbitan monooleate, sorbitan monostearate, or sorbitan monolaurate, but is not limited thereto.

An amino acid sequence represented by a specific sequence number described in the present specification is not limited to a protein (peptide) represented by the specific sequence number, and variants of the amino acid sequence are included in the scope of the present invention as long as functional equivalence is maintained. Specifically, an amino acid sequence having a sequence identity of 80% or more, more preferably 90% or more, and even more preferably 95% or more with an amino acid sequence represented by a specific sequence number may be included. For example, it may include a protein (peptide) having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The “percent sequence identity” of an amino acid is confirmed by comparing a comparison region with an optimally aligned sequence, and in the comparison region, a part of the amino acid sequence may include an addition or deletion (i.e. gap) compared to a reference sequence (including no addition or deletion) for the optimal alignment of two sequences.

A polynucleotide consisting of a base sequence represented by a specific sequence number described in the present specification is not limited to the corresponding base sequence, and variants of the base sequence are included within the scope of the present invention. A nucleic acid molecule of a base sequence of the present invention is a concept including a functional equivalent of the nucleic acid molecule constituting it, for example, variants formed from a nucleic acid molecule in which some base sequences are modified by deletion, substitution, or insertion but are still capable of performing the same function as the nucleic acid. Specifically, a polynucleotide disclosed in the present invention may include a base sequence having a sequence identity of 70% or more, more preferably 80% or more, even more preferably 90% or more, and most preferably 95% or more with a base sequence represented by a specific sequence number. For example, it includes a polynucleotide having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The “percent sequence identity” of a polynucleotide is confirmed by comparing a comparison region between two optimally aligned sequences, and a part of a polynucleotide sequence in the comparison region may include an addition or deletion (i.e. gap) compared to a reference sequence (including no addition or deletion) for the optimal alignment of two sequences.

In the present invention, “pharmaceutical composition” refers to a composition prepared for the purpose of preventing or treating a disease, and it may be formulated and used in various forms according to conventional methods. For example, it may be formulated into oral dosage forms such as powder, granules, tablets, capsules, suspensions, emulsions, and syrup or formulated and used in the form of external preparations, suppositories, and sterile injection solutions.

The pharmaceutical composition according to the present invention may further include appropriate carriers, excipients, and diluents that are commonly used in preparation of pharmaceutical compositions. The excipient may be, for example, one or more selected from the group consisting of diluents, binders, disintegrants, lubricants, adsorbents, humectants, film-coating materials, and controlled-release additives.

The pharmaceutical composition according to the present invention may be formulated and used by conventional methods in the form of powder, granules, sustained-release granules, enteric-coated granules, solutions, eye drops, elixirs, emulsions, suspensions, spirits, troches, perfumes, limonade, tablets, sustained-release tablets, enteric-coated tablets, sublingual tablets, hard capsules, soft capsules, sustained-release capsules, enteric-coated capsules, pills, tinctures, soft extracts, dry extracts, liquid extracts, injections, capsules, perfusates, plasters, lotions, pastes, sprays, inhalants, patches, sterilized injection solutions, or external preparations such as aerosols, and the external preparations may be formulated as creams, gel, patches, sprays, ointments, plasters, lotions, liniments, pastes, or cataplasmas.

Carriers, excipients, and diluents that may be included in the pharmaceutical composition according to the present invention include lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

In the case of formulation, the composition according to the present invention may be prepared using diluents or excipients such as commonly used fillers, extenders, binders, wetting agents, disintegrants, and surfactants.

As additives for tablets, powder, granules, capsules, pills, and troches according to the present invention, excipients such as corn starch, potato starch, wheat starch, lactose, white sugar, glucose, fructose, di-mannitol, precipitated calcium carbonate, synthetic aluminum silicate, calcium hydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethyl cellulose (HPMC) 1928, HPMC 2208, HPMC 2906, HPMC 2910, propylene glycol, casein, calcium lactate, and Primogel; and binders such as gelatin, gum arabic, ethanol, agar powder, cellulose acetate phthalate, carboxymethyl cellulose, calcium carboxymethyl cellulose, glucose, purified water, sodium caseinate, glycerin, stearic acid, sodium carboxymethyl cellulose, sodium methyl cellulose, methyl cellulose, microcrystalline cellulose, dextrin, hydroxycellulose, hydroxypropyl starch, hydroxymethyl cellulose, refined shellac, starch gelatin, hydroxypropyl cellulose, hydroxypropyl methylcellulose, polyvinyl alcohol, and polyvinylpyrrolidone may be used; and disintegrants such as hydroxypropyl methyl cellulose, corn starch, agar powder, methyl cellulose, bentonite, hydroxypropyl starch, sodium carboxymethyl cellulose, sodium alginate, calcium carboxymethyl cellulose, calcium citrate, sodium lauryl sulfate, silicic acid anhydride, 1-hydroxypropyl cellulose, dextran, ion exchange resins, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, white sugar, magnesium aluminum silicate, D-sorbitol solution, and light anhydrous silicic acid; and lubricants such as calcium stearate, magnesium stearate, stearic acid, hydrogenated vegetable oils, talc, Lycopodium, kaolin, Vaseline, sodium stearate, cacao fat, sodium salicylate, magnesium salicylate, polyethylene glycol (PEG) 4000, PEG 6000, liquid paraffin, hydrogen-added soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oils, polyethylene glycol fatty acid ether, sodium chloride, sodium acetate, sodium oleate, DL-leucine, and light anhydrous silicic acid may be used.

As additives for liquid formulations according to the present invention, water, dilute hydrochloric acid, dilute sulfuric acid, sodium citrate, sucrose monostearate, polyoxyethylene sorbitol fatty acid esters (Tween esters), polyoxyethylene monoalkyl ethers, lanolin ethers, lanolin esters, acetic acid, hydrochloric acid, ammonia water, ammonium carbonate, potassium hydroxide, sodium hydroxide, prolamin, polyvinylpyrrolidone, ethyl cellulose, sodium carboxymethyl cellulose or the like may be used.

A solution of white sugar, other sugars, or sweeteners or the like may be used in the syrup according to the present invention, and flavoring agents, colorants, preservatives, stabilizers, suspending agents, emulsifiers, thickening agents or the like may be used as needed.

Purified water may be used in the emulsions according to the present invention, and emulsifiers, preservatives, stabilizers, flavoring agents or the like may be used as needed.

Suspending agents such as acacia, tragacanth, methylcellulose, carboxymethylcellulose, sodium carboxymethylcellulose, microcrystalline cellulose, sodium alginate, HPMC, HPMC 1828, HPMC 2906, HPMC 2910 or the like may be used in the suspensions according to the present invention, and surfactants, preservatives, stabilizers, colorants, and flavoring agents may be used as needed.

The injections according to the present invention may include solvents such as distilled water for injection, 0.9% sodium chloride for injection, Ringer's solution, dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid, benzyl benzoate; solubilizing agents such as sodium benzoate, sodium salicylate, sodium acetate, urea, urethane, monoethyl acetamide, butazolidine, propylene glycol, Tweens, nicotinic acid amide, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediaminetetraacetic acid; antioxidants agents such as sodium bisulfide 0.1%, sodium formaldehyde sulfoxylate, thiourea, disodium ethylenediaminetetraacetate, and acetone sodium bisulfite; analgesics such as benzyl alcohol, chlorobutanol, procaine hydrochloride, glucose, and calcium gluconate; and suspending agents such as carboxymethyl (CM) sodium, sodium alginate, Tween 80, and aluminum monostearate.

In the suppositories according to the present invention, bases such as cacao oil, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G), Cebes Pharma 16, Hexalide Base 95, Cotomar, Hydrocote SP, S-70-XXA, S-70-XX75 (S-70-XX95), Hydrokote 25, Hydrokote 711, Idropostal, Massa estrarium (A, AS, B, C, D, E, I, T), Massa-MF, Massaupol, Masupol-15, Neosupostal-N, Paramount-B, Suposiro (OSI, OSIX, A, B, C, D, H, L), suppositories base type IV (AB, B, A, BC, BBG, E, BGF, C, D, 299), Supostal (N, Es), Wecobi (W, R, S, M, Fs), Tegestor triglyceride base (TG-95, MA, 57) may be used.

Solid preparations for oral administration include tablets, pills, powder, granules, and capsules. These solid preparations are prepared by mixing the extract with at least one excipient, for example, starch, calcium carbonate, sucrose, lactose, gelatin, etc. In addition to simple excipients, lubricants such as magnesium stearate and talc are also used.

Suspensions, oral solutions, emulsions, syrup, and the like correspond to liquid preparations for oral administration, and in addition to the commonly used simple diluents such as water and liquid paraffin, various excipients, for example, wetting agents, sweeteners, flavoring agents, and preservatives may be included. Preparations for parenteral administration include sterilized aqueous solutions, non-aqueous solutions, suspensions, emulsions, freeze-dried preparations, and suppositories. As non-aqueous solvents and suspensions, propylene glycol, polyethylene glycol, vegetable oils such as olive oil, and an injectable ester such as ethyl oleate may be used.

The pharmaceutical composition according to the present invention is administered in a pharmaceutically effective amount. In the present invention, “pharmaceutically effective amount” refers to an amount that is sufficient to treat a disease with a reasonable benefit/risk ratio applicable to medical treatment, and an effective dose level may be determined based on factors including the type and severity of a patient's disease, drug activity, sensitivity to drug, administration time, administration route and excretion rate, treatment duration, and drugs used simultaneously, and other factors well known in the medical field.

The pharmaceutical composition according to the present invention may be administered as an individual therapeutic agent or concomitantly with another therapeutic agent, and it may be administered sequentially or simultaneously with conventional therapeutic agents, and it may be administered once or multiple times. It is important to administer an amount that may achieve the maximum effect with the minimum amount without side effects by considering all of the above factors, and this may be easily determined by those skilled in the art to which the present invention pertains.

The pharmaceutical composition of the present invention may be administered to a subject through various routes. All modes of administration are considered, and it may be administered by, for example, oral administration, subcutaneous injection, intraperitoneal administration, intravenous injection, intramuscular injection, paraspinal space (intrathecal) injection, sublingual administration, buccal administration, intrarectal injection, vaginal injection, ocular administration, auricular administration, nasal administration, inhalation, spraying through the mouth or nose, dermal administration, transdermal administration or the like.

The pharmaceutical composition of the present invention is determined according to the type of drug that is an active ingredient along with various relevant factors such as the disease to be treated, administration route, the patient's age, gender, and weight, and the severity of the disease.

In addition, the present invention provides an alopecia prevention or treatment method, comprising administering a composition comprising the botulinum toxin recombinant protein as an active ingredient to a subject in need thereof.

In addition, the present invention provides a use of a composition comprising the botulinum toxin recombinant protein as an active ingredient for preventing or treating alopecia.

In addition, the present invention provides a use of the botulinum toxin recombinant protein for preparing a drug for treating alopecia.

In addition, the present invention provides a method of ameliorating alopecia, comprising administering a composition comprising the botulinum toxin recombinant protein as an active ingredient to a subject in need thereof.

In addition, the present invention provides a use of a composition comprising the botulinum toxin recombinant protein as an active ingredient for ameliorating alopecia.

In addition, the present invention provides a use of the botulinum toxin recombinant protein for preparing a drug for ameliorating alopecia.

In addition, the present invention provides a method for promoting hair growth comprising administering a composition comprising the botulinum toxin recombinant protein as an active ingredient to a subject in need thereof.

In addition, the present invention provides a use for promoting hair growth of a composition comprising the botulinum toxin recombinant protein as an active ingredient.

In addition, the present invention provides a use of the botulinum toxin recombinant protein for preparing a drug for promoting hair growth.

In the present invention, “subject” refers to a subject that requires treatment of a disease, and more specifically, human or non-human primates, and mammals such as mice, rats, dogs, cats, horses, and cows.

In the present invention, “administration” refers to providing a predetermined amount of the composition of the present invention to a subject by any appropriate method.

In the present invention, “ameliorating” may refer to any action by which alopecia symptoms are changed for the better or beneficially changed by administration of the composition according to the present invention, and may also include preventive or therapeutic actions.

In the present invention, “prevention” refers to all actions that suppress or delay the onset of a target disease, and “treatment” refers to all actions that change for the better or beneficially change a target disease and associated metabolic abnormalities thereof by administration of the pharmaceutical composition according to the present invention.

In the present invention, “treating” may include without limitation any act of administering the botulinum toxin recombinant protein to a subject and allowing a subject to be in contact with the same.

In addition, as another aspect of the present invention, the present invention provides a quasi-drug composition for ameliorating alopecia, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain. The specific details of the botulinum toxin recombinant protein are as described above.

The term “quasi-drug” of the present invention refers to articles with a milder effect than that of pharmaceuticals among articles used for the purpose of diagnosing, treating, ameliorating, alleviating, treating, or preventing diseases in humans or animals. For example, according to the Pharmaceutical Affairs Act, a quasi-drug excludes articles used for pharmaceutical uses and includes products used for treatment or prevention of diseases in humans/animals and products that have a mild or no direct effect on the human body.

When the botulinum toxin recombinant protein according to the present invention is used as a quasi-drug additive, the composition may be added as is or used together with another quasi-drug or quasi-drug component, and may be used appropriately according to conventional methods. The mixing amount of an active ingredient may be appropriately determined according to the purpose of use.

A quasi-drug composition for ameliorating hair loss of the present invention is not particularly limited in dosage forms thereof, and may be formulated in various forms as quasi-drugs known in the art to exhibit an effect of ameliorating alopecia. The above formulated quasi-drugs include hair tonics, hair lotions, hair creams, hair sprays, hair mousse, hair gels, hair conditioners, hair shampoos, hair rinses, hair packs, hair treatments, eyebrow growth agents, eyelash growth agents, eyelash nutritional agents, pet shampoos, pet rinses, hand sanitizers, detergent soaps, soaps, disinfectants, wet wipes, masks, ointments, patches or filter fillers, and include all quasi-drugs in the conventional sense.

In addition, in each formulation, other ingredients may optionally be selected and mixed with a quasi-drug composition for ameliorating alopecia depending on the formulation or purpose of use of other quasi-drugs. The mixing amount of an active ingredient may be appropriately determined depending on the purpose of use and may include, for example, conventional adjuvants such as thickeners, stabilizers, solubilizing agents, vitamins, pigments, and fragrances, and carriers. A quasi-drug composition including the botulinum toxin recombinant protein of the present invention as an active ingredient has almost no toxicity or side effects on cells and thus may be effectively used as a quasi-drug material.

In addition, as still another aspect of the present invention, the present invention provides a composition for external skin application for ameliorating alopecia, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain. The specific details of the botulinum toxin recombinant protein are as described above.

The composition for external skin application of the present invention includes a botulinum toxin recombinant protein as an active ingredient and may include a pharmaceutically acceptable carrier. In addition, the composition for external skin application of the present invention may further include adjuvants commonly used in the field of dermatology such as fatty substances, organic solvents, solubilizing agents, thickening and gelling agents, softeners, antioxidants, suspending agents, stabilizers, foaming agents, flavoring agents, surfactants, water, ionic or non-ionic emulsifiers, fillers, sequestering agents and chelating agents, preservatives, vitamins, blocking agents, wetting agents, essential oils, dyes, pigments, hydrophilic or lipophilic active agents, lipid vesicles or any other ingredients commonly used in external skin preparations. In addition, the ingredients may be introduced in amounts commonly used in the field of dermatology.

Pharmaceutically acceptable carriers in the composition for external skin application of the present invention vary depending on the formulation form, but they may include hydrocarbons such as Vaseline, liquid paraffin, and gelled hydrocarbons (Plastibase); animal and vegetable oils such as medium chain fatty acid triglycerides, pork fat, hard fat, and cocoa fat; higher fatty alcohols and fatty acids, and esters thereof such as cetanol, stearyl alcohol, stearic acid, and isopropyl palmitate; water-soluble bases such as polyethylene glycol, 1,3-butylene glycol, glycerol, gelatin, white sugars, and sugar alcohols; emulsifiers such as glycerin fatty acid ester, polyoxyl stearate, and polyoxyethylene hydrogenated castor oil; adhesives such as acrylic acid ester and sodium alginate; propellants such as liquefied petroleum gas and carbon dioxide; and preservatives such as paraoxybenzoic acid esters. Furthermore, in addition to these, stabilizers, fragrances, colorants, pH adjusters, diluents, surfactants, preservatives, antioxidants, and the like may be added as needed. When the composition for external skin application according to the present invention is used, it is preferable to apply it by a conventional method to the skin of a hair loss area.

The composition for external skin application according to the present invention may be formulated to include a cosmetically or dermatologically acceptable medium or base. This is a formulation suitable for transdermal administration, and the composition for external skin application may be provided in the form of solutions, gels, solids, pasty anhydrous products, emulsions obtained by dispersing an oil phase in an aqueous phase, suspensions, microemulsions, microcapsules, microgranules or ionic (liposome) and non-ionic vesicular dispersant, or in the form of creams, toners, lotions, powder, ointments, sprays, packs, conceal sticks, hair tonic, hair nourishing lotions, hair treatments, hair rinses, hair shampoos, hair lotions or the like. The composition for external skin application may also be used in the form of foam or in the form of an aerosol composition further containing a compressed propellant. These compositions may be prepared according to conventional methods in the art.

The dosage of the composition for external skin application may vary depending on the weight, age, gender, and health conditions of a subject to be administered, administration period, clearance rate, severity of disease and the like.

In addition, as yet another aspect of the present invention, the present invention provides a cosmetic composition for ameliorating alopecia, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain. The specific details of the botulinum toxin recombinant protein are as described above.

Meanwhile, ingredients included in the cosmetic composition of the present invention may include ingredients commonly used in cosmetic compositions in addition to the botulinum toxin recombinant protein of the present invention as an active ingredient, for example, they may include conventional adjuvants such as antioxidants, stabilizers, solubilizing agents, vitamins, pigments, and fragrances, and carriers.

The cosmetic composition of the present invention may be prepared in any formulations commonly prepared in the art, for example, solutions, suspensions, emulsions, pastes, hair gels, hair creams, hair lotions, hair powder, soaps, surfactant-containing shampoos, surfactant-free shampoos, hair oils, hair packs, hair essence, sprays, but is not limited thereto.

When the formulation of a cosmetic composition of the present invention is a paste, cream or gel, animal oil, vegetable oils, wax, paraffin, starch, tragacanth, cellulose derivatives, polyethylene glycol, silicone, bentonite, silica, talc or zinc oxide or the like may be used as a carrier ingredient.

When the formulation of the cosmetic composition of the present invention is a solution or emulsion, a solvent, solubilizing agent, or emulsifying agent is used as a carrier ingredient, and it may include, for example, water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the cosmetic composition of the present invention is a suspension, a liquid diluent such as water, ethanol, and propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth may be used as a carrier ingredient.

When the formulation of the cosmetic composition of the present invention is powder or a spray, lactose, talc, silica, aluminum hydroxide, calcium silicate, or polyamide powder may be used as a carrier ingredient, and in particular, when the formulation is a spray, it may further include a propellent such as chlorofluorohydrocarbon, propane/butane, and dimethyl ether.

When the formulation of the cosmetic composition of the present invention is a surfactant-containing shampoo, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamido betaine, aliphatic alcohols, fatty acid glyceride, fatty acid diethanolamide, vegetable oils, lanolin derivatives, or ethoxylated glycerol fatty acid ester may be used as a carrier ingredient.

When the cosmetic composition of the present invention is in the form of a soap, surfactant-containing shampoo, or surfactant-free shampoo formulation, it can be applied to the skin and then wiped off, removed, or washed with water. As a specific example, the soap includes liquid soaps, powdered soaps, solid soaps, and oil soaps, but is not limited thereto.

### Modes of the Invention

Hereinafter, preferred examples are presented to aid understanding of the present invention. However, the following examples are provided only for easier understanding of the present invention, and the content of the present invention is not limited by the following examples.

### EXAMPLES

### Example 1. Evaluation of Cytotoxicity in Human Follicle Dermal Papilla Cells (HFDPCs)

### 1-1. Cell Line Selection and Cell Culture

To evaluate the cell viability of a skin-penetrating botulinum-derived ingredient peptide (botulinum toxin recombinant protein) prepared according to Korea Patent No. 10-1882461, HFDPCs (PromoCell, Germany) were purchased and used. The HFDPCs were inoculated into each 100 mm² culture dish at 1×10⁶ cells/dish and cultured at 37° C. in an incubator (Sanyo, Japan) containing 5% carbon dioxide after adding a human dermal papilla growth medium containing penicillin (100 IU/ml), streptomycin (100 g/mL), and supplements.

### 1-2. Cell Viability Evaluation Method

The thiazolyl blue tetrazolium bromide (MTT) analysis method is a test method for measuring the ability of mitochondria to reduce MTT tetrazolium, which is a yellow water-soluble substrate, to red-purple formazan (3-(4,5-dimethylthizol-2-yl)-2,5-diphenyl tetrazolium) by the action of cellular dehydrogenase. Since MTT reduction occurs in metabolically active cells, it is widely used to evaluate cytotoxicity and cell viability in cultured cells.

The HFDPC cells subcultured in Example 1-1 were dispensed into a 96-well plate at 1×10⁴ cells/well and cultured for 24 hours in an incubator containing 5% carbon dioxide at 37° C. Next, the HFDPC cells were treated with a skin-penetrating botulinum-derived ingredient peptide at final concentrations of 0.0005, 0.005, 0.05, and 0.5 ppm, and in the positive control group (FBS), cells were treated with fetal bovine serum (FBS) diluted to final concentrations of 15%, 20%, 25%, and 30% and cultured under cell culture conditions for 24 hours.

After culture, the MTT solution was added to each well and allowed to react for two hours under light blocking conditions. Then, the supernatant was removed, the resulting formazan was completely dissolved with dimethyl sulfoxide (DMSO), and the absorbance was measured at 540 nm with a microplate reader (Biotek Synergy-HT, USA).

The cell viability test was repeated three times, and the result values were calculated using Equation 1 and expressed as cell viability compared to the untreated group, and expressed as the average±standard deviation of the three experiments.

$$Cell viability = \frac{Absorbance of the group treated with the sample}{\begin{matrix}Absorbance of the group \\ untreated with the sample\end{matrix}} \times 100 \qquad [ Equation 1 ]$$

The experimental results were analyzed as follows:
(1) The test was repeated three times, and the results were expressed as mean±standard deviation.
(2) The values measured as a result of the cytotoxicity test were analyzed using the Kruskal-Wallis test followed by the Mann-Whitney U test, and then Bonferroni correction was performed (p<0.0125).
(3) The significance of the values measured in the test was confirmed using IBM SPSS statistics version 21.0 with a hypothesized mean difference of 5% (p<0.05).

### 1-3. Results

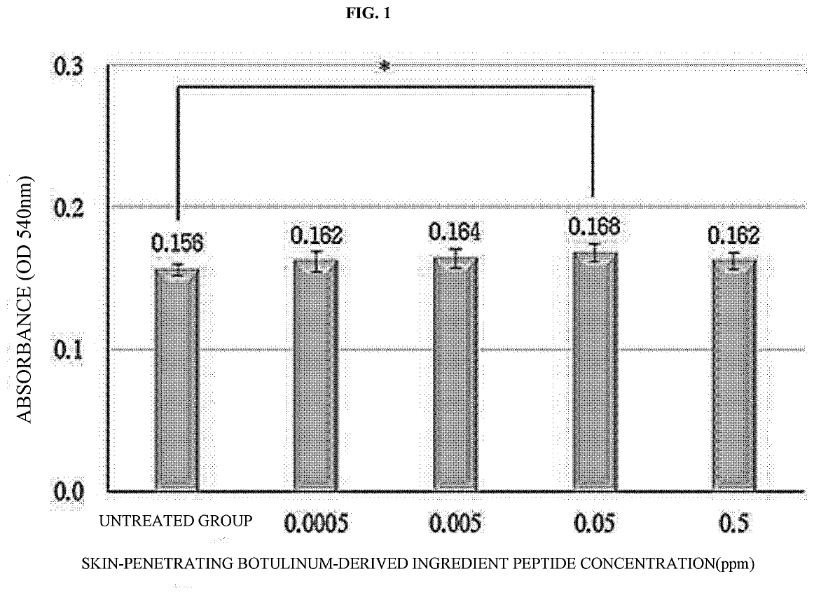

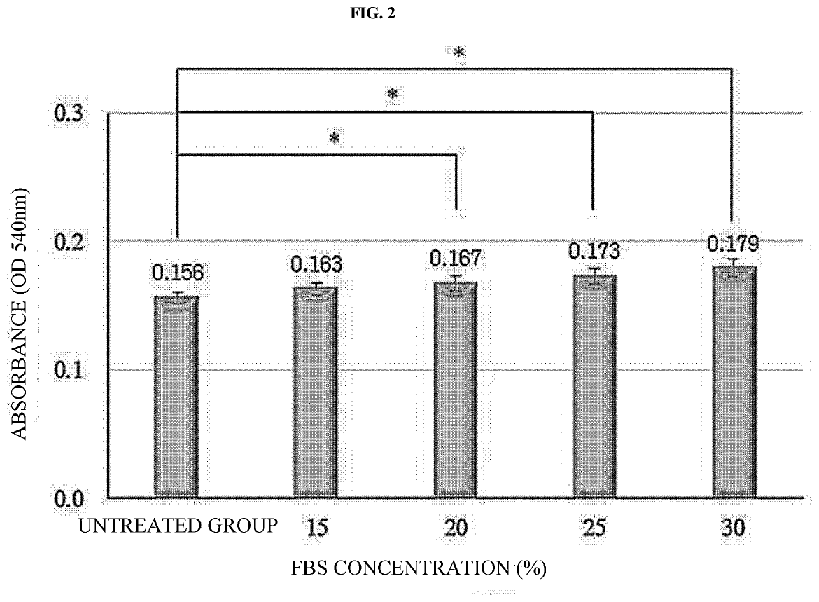

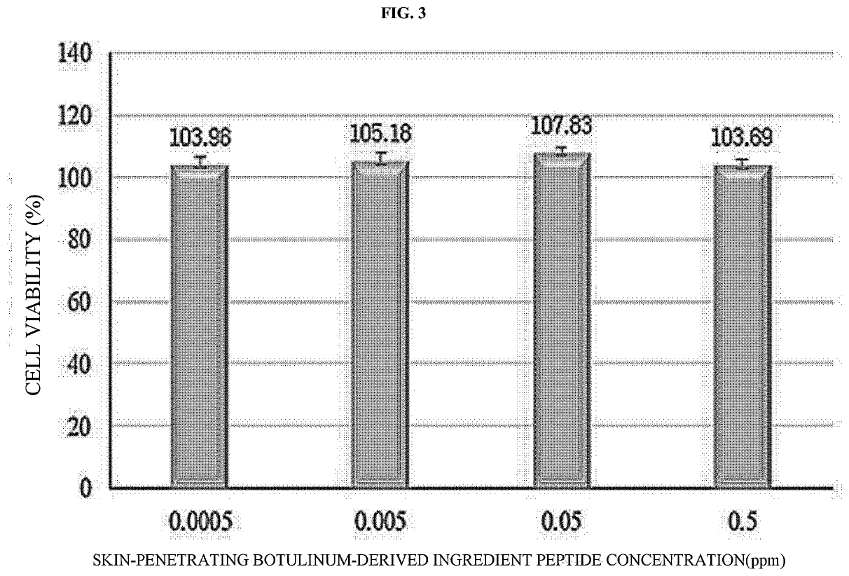

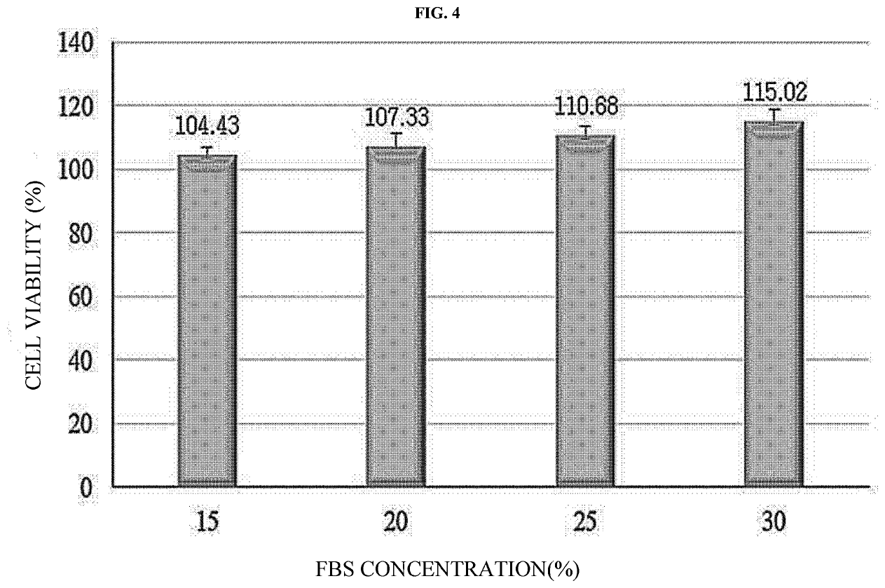

The HFDPC cells were treated with 0.0005, 0.005, 0.05, and 0.5 ppm of the skin-penetrating botulinum-derived ingredient peptide and 15%, 20%, 25%, and 30% of FBS as a positive control, respectively, and the absorbance and cell viability were measured as shown in Tables 1 and 2 and FIGS. 1 to 4.

As a result of the test, the group treated with the skin-penetrating botulinum-derived ingredient peptide showed a cell viability of 103.96% at the 0.0005 ppm concentration, 105.18% at the 0.005 ppm concentration, 107.83% at the 0.05 ppm concentration, and 103.69% at the 0.5 ppm concentration, compared to the untreated group. In the FBS-treated group used as a positive control, the cell viability was 104.43% at the 15% concentration, 107.38% at the 20% concentration, 110.68% at the 25% concentration, and 115.02% at the 30% concentration compared to the untreated group.

In other words, the skin-penetrating botulinum-derived ingredient peptide did not affect cytotoxicity at all concentrations, and at a 0.05 ppm concentration of the skin-penetrating botulinum-derived ingredient peptide, the cell viability significantly increased compared to the untreated group, indicating that the viability of the HFDPCs increased.

TABLE 1

| | | Concentration of skin-penetrating | | | |
|---|---|---|---|---|---|
| | Untreated | botulinum-derived ingredient peptide (ppm) | | | |
| | group^{a} | 0.0005 | 0.005 | 0.05 | 0.5 |
| Absorbance (OD | 0.156 ± | 0.162 ± | 0.164 ± | 0.168 ± | 0.162 ± |
| 540) | 0.004 | 0.007 | 0.007 | 0.006 | 0.006 |
| p-value^{b} | — | 0.050 | 0.019 | <0.001* | 0.050 |
| cell viability (%) | 100 ± | 103.96 ± | 105.18 ± | 107.83 ± | 103.69 ± |
| | 0.00 | 2.51 | 2.80 | 1.79 | 2.16 |

^{a}Untreated group: Group treated with only the culture solution.
^{b}Probability p (Mann-Whitney U test with Bonferroni correction, Significant; *p < 0.0125).

TABLE 2

| | Untreated | Control group (FBS treated group) concentration (%) | | | |
|---|---|---|---|---|---|
| | group^{a} | 15 | 20 | 25 | 30 |
| Absorbance | 0.156 ± | 0.163 ± | 0.167 ± | 0.173 ± | 0.179 ± |
| (OD 540) | 0.004 | 0.005 | 0.006 | 0.006 | 0.007 |
| p-value^{b} | — | 0.014 | <0.001* | <0.001* | <0.001* |
| cell viability | 100 ± | 104.43 ± | 107.33 ± | 110.68 ± | 115.02 ± |
| (%) | 0.00 | 2.07 | 2.35 | 2.32 | 2.62 |

^{a}Untreated group: Group treated with only the culture solution.
^{b}Probability p (Mann-Whitney U test with Bonferroni correction, Significant; *p < 0.0125).

### Example 2. Evaluation of Prostaglandin F₂ₐ Expression

About 30 subtypes of prostaglandins are known to date. In particular, it is known that prostaglandin E₂ and prostaglandin F₂ₐ are mainly distributed in the skin. Prostaglandin F₂ₐ received attention when studies were conducted on the side effects of lengthened eyelashes, increased number of eyelash hairs, and excessive pigmentation in the peripheral regions when a latanoprost eye drop containing a derivative thereof as a main ingredient was used as a therapeutic agent for glaucoma. Afterward, through many studies, Prostaglandin F₂ₐ became known as a factor inducing hair growth, and by checking whether the expression of prostaglandin F₂ₐ increases or decreases according to the treatment of samples, it is used as data to determine the possibility of controlling hair growth and controlling pigmentation.

Accordingly, to evaluate and investigate whether a skin-penetrating botulinum-derived ingredient peptide may induce quantitative changes in prostaglandin F₂ₐ, changes in the expression level of prostaglandin F₂ₓ secreted from keratinocytes were analyzed according to treatment of samples.

2-1. Preparation of samples A skin-penetrating botulinum-derived ingredient peptide at a concentration 2000-fold that of the finished product (0.005 ppm) and/or an alopecia-alleviating functional ingredients (niacinamide+dexapanthenol) at a concentration 200-fold that of the finished product (niacinamide (0.1%) and dexapanthenol (0.2%)) were diluted in purified water and treated in the final cell culture medium at a concentration of 0.2, 0.5, 1, and 2 times the concentration of the finished product, and the control group was treated with purified water in the same amount as the sample treatment amount.

### 2-2. Experimental Method

1) Keratinocytes were inoculated at 3×10^{5/6} wells and cultured for two days.
2) The cells were cultured for one day in a starvation state.
3) The cells were treated with the samples and cultured for one day.
Treated samples:
a) Skin-penetrating botulinum-derived ingredient peptide
b) Skin-penetrating botulinum-derived ingredient peptide+alopecia-alleviating functional ingredient
c) Negative control (COX-2 inhibitor, NS-398)
4) After centrifuging the cell culture medium, the expression level of prostaglandin F₂ₐ was measured using a prostaglandin F₂ₐ assay kit. Specifically, the prostaglandin F₂ₐ test method is a colorimetric method in which absorbance is measured using a spectrophotometer and the results are analyzed. Since the expression of prostaglandin F₂ is highly sensitive to cell conditions and treatment methods, appropriate cell culture and sample treatment methods are important, and standard prostaglandin F₂ₓ should be measured and comparatively analyzed by concentration in each test.
5) The cell activity is measured using the MTT method, and the prostaglandin F₂ₐ values are corrected and analyzed.

### 2-3. Cytotoxicity Test Results

In the cytotoxicity test, measurement was performed by the MTT method. Determination of cytotoxicity is based on the time when cell activity decreases by more than 20% compared to the control group.

Each sample was treated with a cell culture medium at final concentrations of 0.2×, 0.5×, 1×, 2×, and 4×. In the group treated with the skin-penetrating botulinum-derived ingredient peptide alone, no toxicity was observed up to 4×, and in the group treated with the skin-penetrating botulinum-derived ingredient peptide+alopecia-alleviating functional ingredient, strong toxicity was observed starting from the 2× concentration, so the group was excluded from the test groups. The cytotoxicity test results are shown in Table 3.

TABLE 3

| Sample | Treatment | | | | | | % of |
|---|---|---|---|---|---|---|---|
| conditions | concentration | 1 | 2 | 3 | 4 | Mean | control |
| cont | D.W | 0.8770 | 0.8520 | 0.8420 | 0.8480 | 0.8548 | 100 |
| NS-398 | 5 μM | 0.8690 | 0.8540 | 0.8790 | 0.8860 | 0.8720 | 102.0 |
| BDI | 0.2X | 0.8510 | 0.8610 | 0.8800 | 0.8860 | 0.8695 | 101.7 |
| | 0.5X | 0.8540 | 0.8650 | 0.8870 | 0.8840 | 0.8725 | 102.1 |
| | 1X | 0.8540 | 0.8540 | 0.8800 | 0.8820 | 0.8675 | 101.5 |
| | 2X | 0.8580 | 0.8550 | 0.8720 | 0.8720 | 0.8643 | 101.1 |
| | 4X | 0.8780 | 0.8590 | 0.8640 | 0.8710 | 0.8680 | 101.6 |
| BDI + AFI | 0.2X | 0.8790 | 0.8730 | 0.8880 | 0.8890 | 0.8823 | 103.2 |
| | 0.5X | 0.8600 | 0.8620 | 0.8680 | 0.8750 | 0.8663 | 101.3 |
| | 1X | 0.8710 | 0.8790 | 0.8540 | 0.8530 | 0.8643 | 101.1 |

BDI: Botulinum-derived ingredient; AFI: alopecia-alleviating functional ingredient.

### 2-4. Results of Prostaglandin F₂ₐ Analysis Using Spectrophotometer

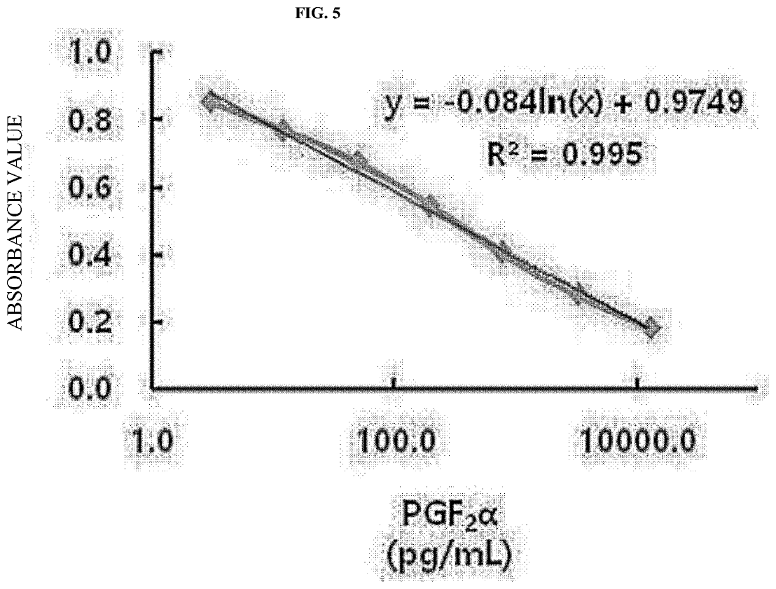

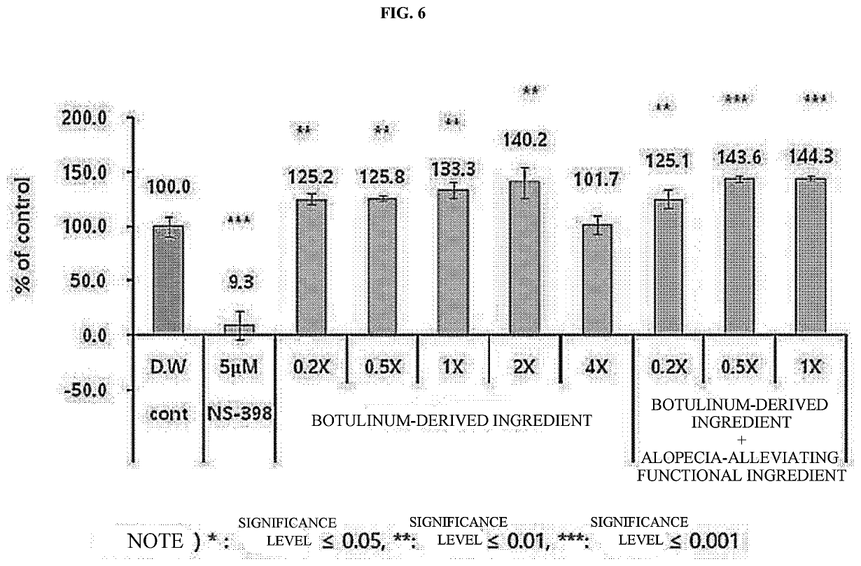

The prostaglandin F₂ analysis using a spectrophotometer is a test method that has the characteristic that the higher the expression level of prostaglandin F₂ₓ, the lower the absorbance value, as shown in the absorbance measured at each concentration of standard prostaglandin F₂ₐ. Specifically, as shown in Table 4 and FIG. 5, a standard curve was obtained by applying standard prostaglandin F₂ₐ according to the amount, and the amount of prostaglandin F₂% was analyzed using this standard curve. This absorbance value was recalibrated to the cell activity value for a final analysis, and the results are shown in Tables 5 and 6 and FIG. 6.

TABLE 4

| PGF_{2α} (pg/mL) | Absorbance value (405 nm) |
|---|---|
| 0 | 0.9360 |
| 3.1 | 0.8570 |
| 12.2 | 0.7710 |
| 48.8 | 0.6750 |
| 195.3 | 0.5470 |
| 781.3 | 0.4060 |
| 3125 | 0.2830 |
| 12500 | 0.1860 |

As a result of the analysis, considering the expression level of prostaglandin F₂ₓ in the control group as 100%, in the group treated with the skin-penetrating botulinum-derived ingredient peptide alone, an increase in expression of 25.2% at the treatment concentration of 0.2×, 25.8% at 0.5×, 33.3% at 1×, and 40.2% at 2× was confirmed. However, at 4×, a decrease in the expression level to the level of the control group was observed. In the combined treatment of the skin-penetrating botulinum-derived ingredient peptide and alopecia-alleviating functional ingredient, an increase in expression of 25.1% at the treatment concentration of 0.2×, 43.4% at 0.5×, and 44.3% at 1× was confirmed.

At this time, n=4 in the test, and as a result of an analysis using the Student's t-test as a statistical technique, it was confirmed that significance was found in all sections where the expression level of prostaglandin F₂ₐ increased.

TABLE 5

| | | Absorbance value | | | | | | | | PGF₂alpha | | | | | |
|---|---|---|---|---|---|---|---|---|---|---|---|---|---|---|---|
| SC | [T] | (405 nm) | | | | PGF₂alpha (pg/mL) | | | | (pg/mL)/MTT | | | | M | SD |
| cont | D.W | 0.4220 | 0.4150 | 0.4160 | 0.4060 | 722.1 | 784.8 | 775.5 | 873.6 | 823.4 | 921.2 | 921.1 | 1030.2 | 923.9 | 84.5 |
| NS- | 5μM | 0.6170 | 0.6000 | 0.6110 | 0.6220 | 70.9 | 86.8 | 76.1 | 66.8 | 81.5 | 101.6 | 86.6 | 75.4 | 86.3 | 11.2 |
| 398 | | | | | | | | | | | | | | | |
| BDI | 0.2X | 0.4010 | 0.3970 | 0.3880 | 0.3910 | 927.2 | 972.4 | 1082.4 | 1044.4 | 1089.5 | 1129.4 | 1230.0 | 1178.8 | 1156.9 | 60.9 |
| | 0.5X | 0.3930 | 0.3970 | 0.3920 | 0.3920 | 1019.8 | 972.4 | 1032.0 | 1032.0 | 1194.2 | 1124.2 | 1163.5 | 1167.5 | 1162.3 | 28.9 |
| | 1X | 0.3920 | 0.3970 | 0.3800 | 0.3880 | 1032.0 | 972.4 | 1190.5 | 1082.4 | 1208.5 | 1138.6 | 1352.9 | 1227.2 | 1231.8 | 89.3 |
| | 2X | 0.3890 | 0.3700 | 0.3940 | 0.3900 | 1069.6 | 1341.0 | 1007.8 | 1056.9 | 1246.6 | 1568.5 | 1155.7 | 1212.0 | 1295.7 | 185.7 |
| | 4X | 0.4190 | 0.4160 | 0.4090 | 0.4040 | 748.3 | 775.5 | 842.9 | 894.6 | 852.3 | 902.8 | 975.6 | 1027.1 | 939.5 | 77.3 |
| BDI + | 0.2X | 0.3950 | 0.4040 | 0.3870 | 0.3870 | 995.8 | 894.6 | 1095.3 | 1095.3 | 1132.9 | 1024.8 | 1233.5 | 1232.1 | 1155.8 | 99.2 |
| AFI | 0.5X | 0.3860 | 0.3850 | 0.3790 | 0.3820 | 1108.4 | 1121.7 | 1204.8 | 1162.5 | 1288.9 | 1301.3 | 1388.0 | 1328.6 | 1326.7 | 44.1 |
| | 1X | 0.3820 | 0.3840 | 0.3820 | 0.3830 | 1162.5 | 1135.2 | 1162.5 | 1148.7 | 1334.7 | 1291.4 | 1361.2 | 1346.7 | 1333.5 | 30.1 |

SC: Sample Conditions; [T]: Treatment Concentration; M: Mean; SD: Standard Deviation; BDI: Botulinum-derived ingredient; AFI: alopecia-alleviating functional ingredient.

TABLE 6

| Sample conditions | Treatment concentration | % of control |
|---|---|---|
| cont. | D.W | 100.0 |
| NS-398 | 5 μM | 9.3 |
| BDI | 0.2X | 125.2 |
| | 0.5X | 125.8 |
| | 1X | 133.3 |
| | 2X | 140.2 |
| | 4X | 101.7 |
| BDI + AFI | 0.2X | 125.1 |
| | 0.5X | 143.6 |
| | 1X | 144.3 |

BDI: Botulinum-derived ingredient; AFI: alopecia-alleviating functional ingredient.

From the above results, it could be confirmed that in both the group treated with the skin-penetrating botulinum-derived ingredient peptide alone and the group treated in combination with the alopecia-alleviating functional ingredient, the expression and secretion of prostaglandin F₂ₐ were induced in keratinocytes.

Since the increase in prostaglandin F₂ₐ expression is related to hair growth, it was determined that applying a skin-penetrating botulinum-derived ingredient peptide to the skin could induce hair growth.

### Example 3. Evaluation of Alopecia-Ameliorating Effect

### 3-1. Experimental Materials and Methods

A total of 4 men (42 to 52 years old) with alopecia symptoms were asked to apply 2.5 cc of a solution containing the skin-penetrating botulinum-derived ingredient peptide (botulinum toxin recombinant protein) directly to the alopecia area every morning, and the conditions of the subjects' scalp was confirmed by taking photos before use, after 8 weeks of use, and after 16 weeks of use. The subjects were asked to evaluate the amelioration of alopecia symptoms as no effect (0)/good (1)/satisfied (2)/very satisfied (3).

In addition, to objectify the experimental results, two evaluators were asked to evaluate the amelioration of alopecia symptoms as no amelioration (0)/slight amelioration (1)/amelioration (2)/excellent amelioration (3).

Furthermore, after 16 weeks of use, a survey was conducted with the subjects about changes in hair thickness, number of hairs lost, reduction in scalp oiliness, cleanliness around hair roots (reduction in sebum, etc.), and hair sinking in the afternoon.

### 3-2. Results

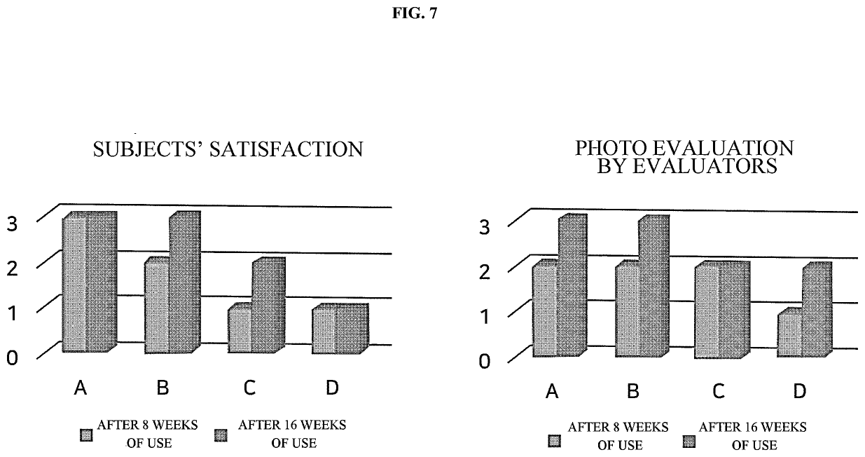

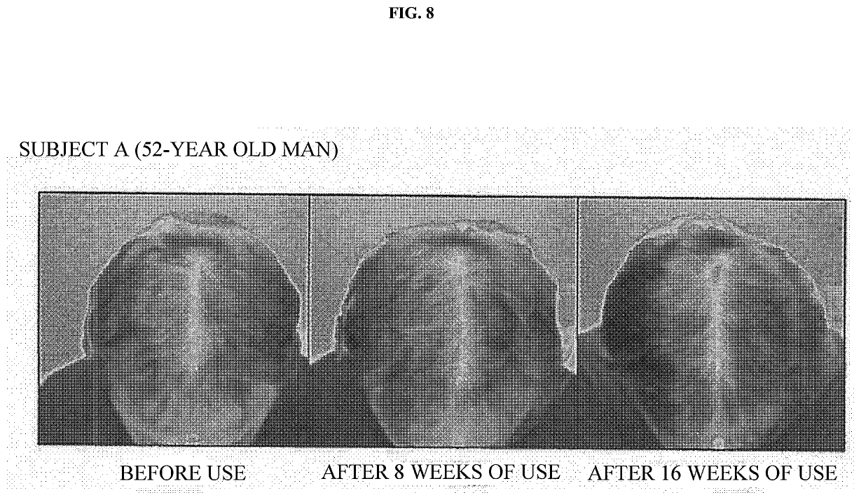

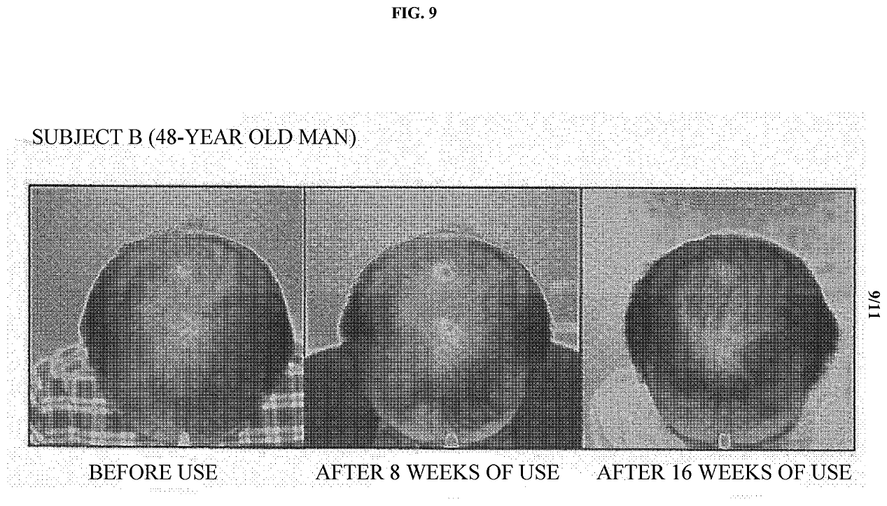

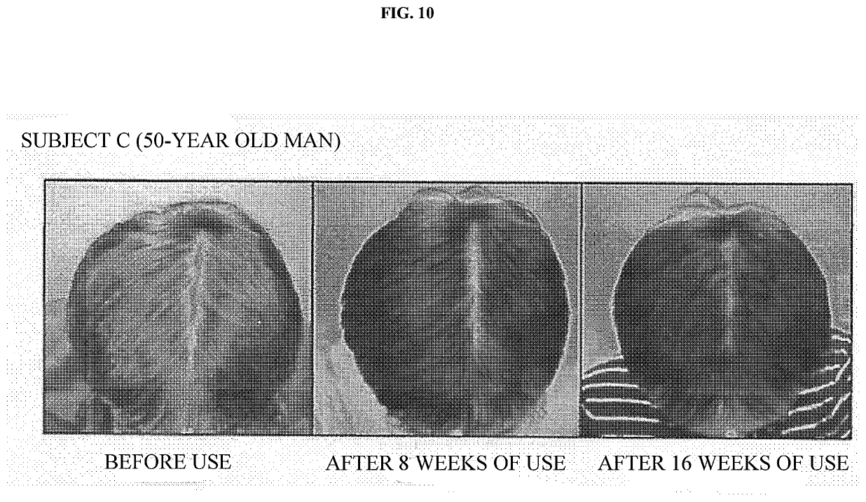

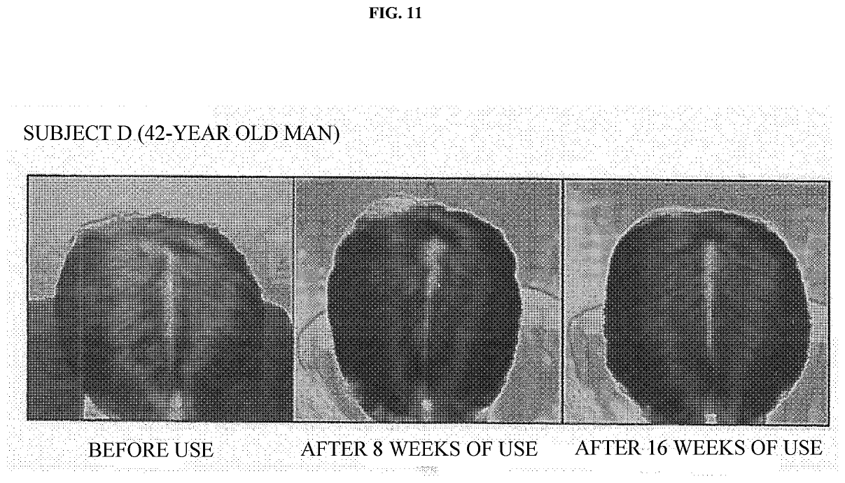

As a result of evaluating the alopecia-ameliorating effect of the skin-penetrating botulinum-derived ingredient peptide by the subjects and evaluators, as shown in Table 7 and FIG. 7, the satisfaction of the subjects increased as the period of use increased, and both evaluators evaluated that the subjects' alopecia symptoms were significantly improved. No particular side effects were found in any of the subjects during the 16-week experiment period. FIGS. 8 to 11 show changes in the subjects' scalp.

TABLE 7

| | Subjects' satisfaction | | Photo evaluation by evaluators | |
|---|---|---|---|---|
| | After 8 weeks of | After 16 weeks | After 8 weeks of | After 16 weeks |
| | use | of use | use | of use |
| Subject A | 3 | 3 | 2 | 3 |
| Subject B | 2 | 3 | 2 | 3 |
| Subject C | 1 | 2 | 2 | 2 |
| Subject D | 1 | 1 | 1 | 2 |

*Subjects' satisfaction: 0 (no effect)/1 (good)/2 (satisfied)/3 (very satisfied).
**Photo evaluation by evaluators: 0 (no amelioration)/1 (slight amelioration)/2 (amelioration)/3 (excellent amelioration).

In addition, as shown in Table 8, in the survey administered to the subjects after 16 weeks of use, overall improvement was found in the subjects' hair thickness, number of hairs lost, reduction in oiliness of the scalp, cleanliness around the hair roots (reduction of sebum, etc.), and hair sinking in the afternoon.

TABLE 8

| Changes in hair | | Slightly | | Hairs thickened |
|---|---|---|---|---|
| thickness | No change | thickened | Much thickened | and strengthened |
| Number of | | 2 | 1 | 1 |
| respondents | | | | |

| Number of hairs | | | | Almost no hair |
|---|---|---|---|---|
| lost | No change | Slightly reduced | Much reduced | lost |
| Number of | | | 3 | 1 |
| respondents | | | | |

| Reduction in | | | | Scalp and hairs |
|---|---|---|---|---|
| oiliness of the | | | | remaining dry in |
| scalp | Slightly reduced | Reduced | Much reduced | the afternoon |
| Number of | 1 | 1 | | 2 |
| respondents | | | | |
| Cleanliness | | | | |
| around the hair | | | | Remain clean all |
| roots | Slightly reduced | Reduced | Much reduced | day long |
| Number of | | 3 | 1 | |
| respondents | | | | |

| Hair sinking in | Slightly | | | No sinking all |
|---|---|---|---|---|
| the afternoon | improved | Improved | Much improved | day long |
| Number of | 2 | | 1 | 1 |
| respondents | | | | |

The description of the present invention described above is for illustrative purposes, and those skilled in the art will understand that the present invention can be easily modified into other specific forms without changing the technical idea or essential features of the present invention. Therefore, the examples described above should be understood in all respects as illustrative and not restrictive.

### INDUSTRIAL APPLICABILITY

Since the botulinum toxin recombinant protein according to the present invention can be easily delivered transdermally through fusion with a cell-penetrating peptide and promote proliferation of dermal papilla cells and enhance prostaglandin F₂ₓ expression to reduce hair loss and promote hair growth, thereby ameliorating alopecia, it can be effectively used for preventing, ameliorating or treating alopecia in fields such as cosmetics and pharmaceuticals. Therefore, the present invention has industrial applicability.

1. An alopecia prevention or treatment method, comprising administering a therapeutically effective amount of a composition comprising a botulinum toxin recombinant protein as an active ingredient to a subject in need thereof,
wherein in the botulinum toxin recombinant protein, a cell-penetrating peptide consisting of an amino acid sequence of SEQ ID NO: 1 is fused to one end or both ends of a botulinum toxin light chain.

2. The method according to claim 1, wherein the botulinum toxin recombinant protein consists of one or more amino acid sequences selected from the group consisting of SEQ ID NO: 31 to SEQ ID NO: 58.

3. The method according to claim 1, wherein the botulinum toxin light chain consists of one or more amino acid sequences selected from the group consisting of SEQ ID NO: 3 to SEQ ID NO: 9.

4. The method according to claim 1, wherein the botulinum toxin light chain further includes a hexahistidine tag at one end.

5. The method according to claim 1, wherein the botulinum toxin light chain is selected from the group consisting of botulinum toxin serotypes A, B, C, D, E, F, and G.

6. The method according to claim 1, wherein the cell-penetrating peptide is fused to a carboxyl terminus, an amino terminus, or both of the botulinum toxin light chain.

7. The method according to claim 1, wherein the fusion is achieved by a peptide bond or a covalent bond.

8. The method according to claim 1, wherein the composition promotes the proliferation of dermal papilla cells.

9. The method according to claim 1, wherein the composition promotes prostaglandin F₂ₐ expression.

10. The method according to claim 1, wherein the composition promotes hair growth and reduces alopecia.

11. The method according to claim 1, wherein the composition is for transdermal administration.

12-17. (canceled)

18. The method according to claim 1, wherein the composition is a pharmaceutical composition, a quasi-drug composition, a composition for external skin application, or a cosmetic composition.


SEQUENCE LISTING (86 sequences)

SEQ ID NO: 1 - protein, 13 aa, synthetic construct
KAMININKFLNQC

SEQ ID NO: 2 - DNA, 39 bp, synthetic construct
  source 1..39: mol_type = other DNA
aaggcgatgataaacataaacaagttcctgaaccagtgc

SEQ ID NO: 3 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 4 - protein, 441 aa, synthetic construct
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 5 - protein, 449 aa, synthetic construct
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 6 - protein, 442 aa, synthetic construct
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 7 - protein, 422 aa, synthetic construct
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 8 - protein, 436 aa, synthetic construct
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 9 - protein, 441 aa, synthetic construct
MPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 10 - DNA, 1347 bp, synthetic construct
  source 1..1347: mol_type = other DNA
atgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtga

SEQ ID NO: 11 - DNA, 1323 bp, synthetic construct
  source 1..1323: mol_type = other DNA
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaa

SEQ ID NO: 12 - DNA, 1347 bp, synthetic construct
  source 1..1347: mol_type = other DNA
atgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaa

SEQ ID NO: 13 - DNA, 1326 bp, synthetic construct
  source 1..1326: mol_type = other DNA
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaa

SEQ ID NO: 14 - DNA, 1266 bp, synthetic construct
  source 1..1266: mol_type = other DNA
atgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataagg

SEQ ID NO: 15 - DNA, 1308 bp, synthetic construct
  source 1..1308: mol_type = other DNA
atgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaa

SEQ ID NO: 16 - DNA, 1326 bp, synthetic construct
  source 1..1326: mol_type = other DNA
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaa

SEQ ID NO: 17 - protein, 468 aa, synthetic construct
MGSSHHHHHHSSGLVPRGSHMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 18 - protein, 461 aa, synthetic construct
MGSSHHHHHHSSGLVPRGSHMPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 19 - protein, 469 aa, synthetic construct
MGSSHHHHHHSSGLVPRGSHMPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 20 - protein, 462 aa, synthetic construct
MGSSHHHHHHSSGLVPRGSHMTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 21 - protein, 442 aa, synthetic construct
MGSSHHHHHHSSGLVPRGSHMPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 22 - protein, 456 aa, synthetic construct
MGSSHHHHHHSSGLVPRGSHMPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 23 - protein, 461 aa, synthetic construct
MGSSHHHHHHSSGLVPRGSHMPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 24 - DNA, 1437 bp, synthetic construct
  source 1..1437: mol_type = other DNA
atgggcagcagccatcatcatcatcatcacagcagcggcctggtgccgcgcggcagccatatgatgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgactcgagcaccaccaccaccaccactga

SEQ ID NO: 25 - DNA, 1413 bp, synthetic construct
  source 1..1413: mol_type = other DNA
atgggcagcagccatcatcatcatcatcacagcagcggcctggtgccgcgcggcagccatatgatgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaactcgagcaccaccaccaccaccactga

SEQ ID NO: 26 - DNA, 1437 bp, synthetic construct
  source 1..1437: mol_type = other DNA
atgggcagcagccatcatcatcatcatcacagcagcggcctggtgccgcgcggcagccatatgatgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaactcgagcaccaccaccaccaccactga

SEQ ID NO: 27 - DNA, 1416 bp, synthetic construct
  source 1..1416: mol_type = other DNA
atgggcagcagccatcatcatcatcatcacagcagcggcctggtgccgcgcggcagccatatgatgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaactcgagcaccaccaccaccaccactga

SEQ ID NO: 28 - DNA, 1356 bp, synthetic construct
  source 1..1356: mol_type = other DNA
atgggcagcagccatcatcatcatcatcacagcagcggcctggtgccgcgcggcagccatatgatgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggctcgagcaccaccaccaccaccactga

SEQ ID NO: 29 - DNA, 1398 bp, synthetic construct
  source 1..1398: mol_type = other DNA
atgggcagcagccatcatcatcatcatcacagcagcggcctggtgccgcgcggcagccatatgatgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaactcgagcaccaccaccaccaccactga

SEQ ID NO: 30 - DNA, 1416 bp, synthetic construct
  source 1..1416: mol_type = other DNA
atgggcagcagccatcatcatcatcatcacagcagcggcctggtgccgcgcggcagccatatgatgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaactcgagcaccaccaccaccaccactga

SEQ ID NO: 31 - protein, 461 aa, synthetic construct
MKAMININKFLNQCPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 32 - protein, 454 aa, synthetic construct
MKAMININKFLNQCPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 33 - protein, 462 aa, synthetic construct
MKAMININKFLNQCPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 34 - protein, 455 aa, synthetic construct
MKAMININKFLNQCTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 35 - protein, 435 aa, synthetic construct
MKAMININKFLNQCPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 36 - protein, 449 aa, synthetic construct
MKAMININKFLNQCPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 37 - protein, 454 aa, synthetic construct
MKAMININKFLNQCPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 38 - protein, 461 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKCQNLFKNINIMAK

SEQ ID NO: 39 - protein, 454 aa, synthetic construct
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKCQNLFKNINIMAK

SEQ ID NO: 40 - protein, 462 aa, synthetic construct
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKCQNLFKNINIMAK

SEQ ID NO: 41 - protein, 455 aa, synthetic construct
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKCQNLFKNINIMAK

SEQ ID NO: 42 - protein, 435 aa, synthetic construct
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRCQNLFKNINIMAK

SEQ ID NO: 43 - protein, 449 aa, synthetic construct
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKCQNLFKNINIMAK

SEQ ID NO: 44 - protein, 454 aa, synthetic construct
MPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKCQNLFKNINIMAK

SEQ ID NO: 45 - protein, 469 aa, synthetic construct
MKAMININKFLNQCPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKLEHHHHHH

SEQ ID NO: 46 - protein, 462 aa, synthetic construct
MKAMININKFLNQCPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKLEHHHHHH

SEQ ID NO: 47 - protein, 470 aa, synthetic construct
MKAMININKFLNQCPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKLEHHHHHH

SEQ ID NO: 48 - protein, 463 aa, synthetic construct
MKAMININKFLNQCTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKLEHHHHHH

SEQ ID NO: 49 - protein, 443 aa, synthetic construct
MKAMININKFLNQCPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRLEHHHHHH

SEQ ID NO: 50 - protein, 457 aa, synthetic construct
MKAMININKFLNQCPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKLEHHHHHH

SEQ ID NO: 51 - protein, 462 aa, synthetic construct
MKAMININKFLNQCPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKLEHHHHHH

SEQ ID NO: 52 - protein, 481 aa, synthetic construct
MGSSHHHHHHSSGLVPRGSHMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKCQNLFKNINIMAK

SEQ ID NO: 53 - protein, 474 aa, synthetic construct
MGSSHHHHHHSSGLVPRGSHMPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKCQNLFKNINIMAK

SEQ ID NO: 54 - protein, 482 aa, synthetic construct
MGSSHHHHHHSSGLVPRGSHMPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKCQNLFKNINIMAK

SEQ ID NO: 55 - protein, 475 aa, synthetic construct
MGSSHHHHHHSSGLVPRGSHMTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKCQNLFKNINIMAK

SEQ ID NO: 56 - protein, 455 aa, synthetic construct
MGSSHHHHHHSSGLVPRGSHMPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRCQNLFKNINIMAK

SEQ ID NO: 57 - protein, 469 aa, synthetic construct
MGSSHHHHHHSSGLVPRGSHMPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKCQNLFKNINIMAK

SEQ ID NO: 58 - protein, 474 aa, synthetic construct
MGSSHHHHHHSSGLVPRGSHMPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKCQNLFKNINIMAK

SEQ ID NO: 59 - DNA, 1386 bp, synthetic construct
  source 1..1386: mol_type = other DNA
atgaaggccatgatcaatattaacaagttcttaaatcaatgtccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtga

SEQ ID NO: 60 - DNA, 1362 bp, synthetic construct
  source 1..1362: mol_type = other DNA
atgaaggccatgatcaatattaacaagttcttaaatcaatgtccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaa

SEQ ID NO: 61 - DNA, 1386 bp, synthetic construct
  source 1..1386: mol_type = other DNA
atgaaggccatgatcaatattaacaagttcttaaatcaatgtccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaa

SEQ ID NO: 62 - DNA, 1365 bp, synthetic construct
  source 1..1365: mol_type = other DNA
atgaaggccatgatcaatattaacaagttcttaaatcaatgtacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaa

SEQ ID NO: 63 - DNA, 1305 bp, synthetic construct
  source 1..1305: mol_type = other DNA
atgaaggccatgatcaatattaacaagttcttaaatcaatgtccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataagg

SEQ ID NO: 64 - DNA, 1347 bp, synthetic construct
  source 1..1347: mol_type = other DNA
atgaaggccatgatcaatattaacaagttcttaaatcaatgtccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaa

SEQ ID NO: 65 - DNA, 1365 bp, synthetic construct
  source 1..1365: mol_type = other DNA
atgaaggccatgatcaatattaacaagttcttaaatcaatgtccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaa

SEQ ID NO: 66 - DNA, 1386 bp, synthetic construct
  source 1..1386: mol_type = other DNA
atgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgatgtcaaaatttattcaagaacattaatatcatggccaag

SEQ ID NO: 67 - DNA, 1362 bp, synthetic construct
  source 1..1362: mol_type = other DNA
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaatgtcaaaatttattcaagaacattaatatcatggccaag

SEQ ID NO: 68 - DNA, 1386 bp, synthetic construct
  source 1..1386: mol_type = other DNA
atgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaatgtcaaaatttattcaagaacattaatatcatggccaag

SEQ ID NO: 69 - DNA, 1365 bp, synthetic construct
  source 1..1365: mol_type = other DNA
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaatgtcaaaatttattcaagaacattaatatcatggccaag

SEQ ID NO: 70 - DNA, 1305 bp, synthetic construct
  source 1..1305: mol_type = other DNA
atgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggtgtcaaaatttattcaagaacattaatatcatggccaag

SEQ ID NO: 71 - DNA, 1347 bp, synthetic construct
  source 1..1347: mol_type = other DNA
atgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaatgtcaaaatttattcaagaacattaatatcatggccaag

SEQ ID NO: 72 - DNA, 1365 bp, synthetic construct
  source 1..1365: mol_type = other DNA
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaatgtcaaaatttattcaagaacattaatatcatggccaag

SEQ ID NO: 73 - DNA, 1413 bp, synthetic construct
  source 1..1413: mol_type = other DNA
atgaaggccatgatcaatattaacaagttcttaaatcaatgtccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgactcgagcaccaccaccaccaccactga

SEQ ID NO: 74 - DNA, 1389 bp, synthetic construct
  source 1..1389: mol_type = other DNA
atgaaggccatgatcaatattaacaagttcttaaatcaatgtccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaactcgagcaccaccaccaccaccactga

SEQ ID NO: 75 - DNA, 1413 bp, synthetic construct
  source 1..1413: mol_type = other DNA
atgaaggccatgatcaatattaacaagttcttaaatcaatgtccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaactcgagcaccaccaccaccaccactga

SEQ ID NO: 76 - DNA, 1392 bp, synthetic construct
  source 1..1392: mol_type = other DNA
atgaaggccatgatcaatattaacaagttcttaaatcaatgtacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaactcgagcaccaccaccaccaccactga

SEQ ID NO: 77 - DNA, 1332 bp, synthetic construct
  source 1..1332: mol_type = other DNA
atgaaggccatgatcaatattaacaagttcttaaatcaatgtccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggctcgagcaccaccaccaccaccactga

SEQ ID NO: 78 - DNA, 1374 bp, synthetic construct
  source 1..1374: mol_type = other DNA
atgaaggccatgatcaatattaacaagttcttaaatcaatgtccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaactcgagcaccaccaccaccaccactga

SEQ ID NO: 79 - DNA, 1392 bp, synthetic construct
  source 1..1392: mol_type = other DNA
atgaaggccatgatcaatattaacaagttcttaaatcaatgtccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaactcgagcaccaccaccaccaccactga

SEQ ID NO: 80 - DNA, 1446 bp, synthetic construct
  source 1..1446: mol_type = other DNA
atgggcagcagccatcatcatcatcatcacagcagcggcctggtgccgcgcggcagccatatgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgatgtcaaaatttattcaagaacattaatatcatggccaag

SEQ ID NO: 81 - DNA, 1422 bp, synthetic construct
  source 1..1422: mol_type = other DNA
atgggcagcagccatcatcatcatcatcacagcagcggcctggtgccgcgcggcagccatatgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaatgtcaaaatttattcaagaacattaatatcatggccaag

SEQ ID NO: 82 - DNA, 1446 bp, synthetic construct
  source 1..1446: mol_type = other DNA
atgggcagcagccatcatcatcatcatcacagcagcggcctggtgccgcgcggcagccatatgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaatgtcaaaatttattcaagaacattaatatcatggccaag

SEQ ID NO: 83 - DNA, 1425 bp, synthetic construct
  source 1..1425: mol_type = other DNA
atgggcagcagccatcatcatcatcatcacagcagcggcctggtgccgcgcggcagccatatgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaatgtcaaaatttattcaagaacattaatatcatggccaag

SEQ ID NO: 84 - DNA, 1365 bp, synthetic construct
  source 1..1365: mol_type = other DNA
atgggcagcagccatcatcatcatcatcacagcagcggcctggtgccgcgcggcagccatatgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggtgtcaaaatttattcaagaacattaatatcatggccaag

SEQ ID NO: 85 - DNA, 1407 bp, synthetic construct
  source 1..1407: mol_type = other DNA
atgggcagcagccatcatcatcatcatcacagcagcggcctggtgccgcgcggcagccatatgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaatgtcaaaatttattcaagaacattaatatcatggccaag

SEQ ID NO: 86 - DNA, 1425 bp, synthetic construct
  source 1..1425: mol_type = other DNA
atgggcagcagccatcatcatcatcatcacagcagcggcctggtgccgcgcggcagccatatgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaatgtcaaaatttattcaagaacattaatatcatggccaag